(19) United States
(12) Patent Application Publication
(10) Pub. No.: US 2013/0267887 A1
(43) Pub. Date: Oct. 10, 2013

(54) AQUEOUS HUMOR MICRO-BYPASS SHUNTS

(71) Applicants: Malik Kahook, Denver, CO (US); Naresh Mandava, Denver, CO (US); Robin Shandas, Boulder, CO (US); Bryan Rech, Boulder, CO (US)

(72) Inventors: Malik Kahook, Denver, CO (US); Naresh Mandava, Denver, CO (US); Robin Shandas, Boulder, CO (US); Bryan Rech, Boulder, CO (US)

(73) Assignee: THE REGENTS OF THE UNIVERSITY OF COLORADO, Denver, CO (US)

(21) Appl. No.: 13/825,495

(22) PCT Filed: Sep. 21, 2011
(86) PCT No.: PCT/US11/52634

§ 371 Date: Jun. 26, 2013

Related U.S. Application Data
(60) Provisional application No. 61/384,843, filed on Sep. 21, 2010.

(51) Int. Cl. A61F 9/007; B29C 53/02

(52) U.S. Cl. CPC A61F 9/00781; B29C 53/02
USPC 604/9; 604/8; 264/320; 264/241

(57) ABSTRACT

A small shunt can be placed within the eye to aid in drainage of aqueous humor from the anterior chamber of the eye to a pocket between the conjunctiva and the sclera to be absorbed, or to secrete through the cornea or the sclera external to the eye for glaucoma or ocular hypertension treatment. This drainage can decrease the pressure of the eye and potentially modify the course of advancing glaucomatous optic neuropathy as it relates to eye pressure. The shunt is formed of a shape memory polymer material and deformed into a smaller form factor to reduce trauma to the eye resulting from the insertion of the shunt through the sclera to the anterior chamber. Once in situ, the shunt deploys in response to body heat or other external stimulus and expands to its original, larger form factor to provide a secure friction fit of the shunt within the scleral tissue and to enlarge the lumen of the shunt to allow for aqueous flow.

### CROSS REFERENCE TO RELATED APPLICATIONS

This application claims the benefit of priority pursuant to 35 U.S.C. §119(e) of U.S. provisional application No. 61/384,843 filed 21 Sep. 2010 entitled “Aqueous humor micro-bypass shunt,” which is hereby incorporated herein by reference in its entirety for the purposes of PCT Rule 20.6.

The present application is related to the following applications: U.S. patent application Ser. No. 12/295,594 filed 30 Sep. 2008 entitled “Shape memory polymer medical devices”; Patent Cooperation Treaty Application No. PCT/US2006/060297 filed 27 Oct. 2006 entitled “A polymer formulation, a method of determining a polymer formulation, and a method of determining a polymer fabrication”; and U.S. patent application Ser. No. 12/988,983, filed 5 Jan. 2011 entitled “Thiol-vinyl and thiol-yne systems for shape memory polymers,” each of which is hereby incorporated herein by reference in its entirety.

### TECHNICAL FIELD

The technology described herein relates to stent or shunt devices for the treatment of glaucoma or ocular hypertension or for the delivery of drugs to intraocular structures.

### BACKGROUND

Glaucoma is a group of eye diseases that causes pathological changes in the optic disk and corresponding visual field loss resulting in blindness if untreated. Glaucoma is a leading cause of irreversible blindness in the world. It is estimated that 70 million people worldwide have glaucoma, and that nearly 7 million are bilaterally blind from this disease. Millions of people suffer from glaucoma, and it is the third most common reason for adults to visit a medical doctor. Elevated intraocular pressure is an outstanding risk factor for the development of glaucoma and one reason for progression of the disease. Decreasing intraocular pressure is one known modifiable risk factor for glaucoma. Accordingly, treatment of glaucoma has been focused on lowering the intraocular pressure of the fluid in the affected eye.

The aqueous humour is a transparent, watery substance filling the anterior chamber, i.e., the space between the lens and the cornea. The aqueous humor maintains the intraocular pressure. The aqueous humor is constantly secreted by the ciliary processes posterior to the iris, so there is a continuous flow of the aqueous humor from the ciliary processes to the anterior chamber. In a healthy eye, the tissue of the trabecular meshwork allows the aqueous humor to pass through and enter Schlemm's canal, which then empties into aqueous collector channels in the anterior wall of Schlemm's canal and finally into aqueous veins. The trabecular meshwork is located between the iris and cornea. The pressure of the eye is determined by a balance between the production of aqueous humor and its exit through the trabecular meshwork and Schlemm's canal.

Glaucoma is grossly classified into two categories: closed-angle glaucoma and open-angle glaucoma. The closed-angle glaucoma is caused by closure of the anterior angle by contact between the iris and the inner surface of the trabecular meshwork. Closure of this anatomical angle prevents normal drainage of aqueous humor from the anterior chamber of the eye and thus an associated elevation in intraocular pressure that ultimately damages the optic nerve. Open-angle glaucoma is any glaucoma in which the angle of the anterior chamber remains open, but the exit of aqueous through the trabecular meshwork is diminished. The exact cause for diminished filtration is unknown for most cases of open-angle glaucoma. However, there are secondary open-angle glaucomas which may include edema or swelling of the trabecular spaces (from steroid use), abnormal pigment dispersion, or diseases such as hyperthyroidism that produce vascular congestion.

Current therapies for glaucoma are directed at decreasing intraocular pressure. This may initially be by medical therapy with drops or pills that reduce the production of aqueous humor or increase the outflow of aqueous. However, these various drug therapies for glaucoma are sometimes associated with significant side effects, such as foreign body sensation, dry eye syndrome, headache, blurred vision, allergic reactions, respiratory and/or cardiovascular complications and potential interactions with other drugs. When the drug therapy fails, surgical intervention is used. Surgery for open-angle glaucoma consists of laser (trabeculoplasty), trabeculectomy and aqueous shunting implants after failure of trabeculectomy or if trabeculectomy is unlikely to succeed. Trabeculectomy is the surgery that is most widely used and is augmented with topically applied antifibrotic drugs such as 5-fluorouracil or mitomycin-c to decrease scarring and increase surgical success.

Approximately 50,000-60,000 trabeculectomies are performed on Medicare age patients per year in the United States. The number of surgical interventions would likely increase if the morbidity associated with trabeculectomy could be decreased. The current morbidity associated with trabeculectomy consists of failure (10-15%), infection (a life-long risk about 2-5%), choroidal hemorrhage (1%, a severe internal hemorrhage from pressure too low resulting in visual loss), cataract formation, and hypotony maculopathy (potentially reversible visual loss from pressure too low) among others.

By bypassing the local resistance to outflow of aqueous at the point of the resistance and using existing outflow mechanisms, e.g., aqueous veins, surgical morbidity may be greatly decreased. Trabecular bypass surgery may provide much lower risk of hypotony maculopathy, choroidal hemorrhage, infection, and uses existing physiologic outflow mechanisms. This surgery may be performed under topical anesthesia in a physician's office with rapid visual recovery. Even so, the present trabecular bypass devices are larger in size in order to be effective, which can cause significant trauma to the eye and still often result in hemorrhage and infection after placement.

The information included in this Background section of the specification, including any references cited herein and any description or discussion thereof, is included for technical reference purposes only and is not to be regarded subject matter by which the scope of the invention as defined in the claims is to be bound.

### SUMMARY

A shunt formed of shape memory polymer (SMP) material can be stored in a deformed configuration of a smaller form factor and at temperature below the transition temperature Tg. The small form factor shunt can then be inserted into an incision in the eye. Thereafter the body's thermal energy can heat the shunt causing the stent to activate, change shape, and expand. In some embodiments, in the stored configuration the shunt can be compressed such that the lumen is substantially or completely collapsed. In such a state, the inside and outside diameters are smaller than in the activated state. Upon activation, by body heat, the inside and outside diameters will expand. The outside diameter of the shunt can then fit securely within and make a seal with tissue walls surrounding the incision, providing a tight closure. The interior lumen, once opened upon deployment, conducts fluid from the anterior chamber to a pocket between the conjunctiva and sclera for absorption by the aqueous veins or secretion outside the eye. A number of benefits can be derived from this activated state including controlled egress of fluid, decreased chance of hypotony in the short-term, and potential quicker wound healing and visual recovery after eye surgery.

In one implementation, a shunt is disclosed for implantation within the sclera between the aqueous veins and the anterior chamber for treatment of glaucoma. The shunt may be composed of a tube defining a lumen formed of a shape memory polymer material with a glass transition temperature at or slightly above body temperature that has an initial shape and a deformed shape. The shunt has a first lumen diameter and a first outer diameter in the deformed shape. The shunt has a second lumen diameter and a second outer diameter in the initial shape. The second lumen diameter is greater than the first lumen diameter and the second outer diameter is greater than the first outer diameter. The deformed shape is radially or otherwise compressed in the predeployed configuration. The initial configuration is substantially identical to a post implantation, radially expanded configuration. In an alternate or additional embodiment, the shunt may be formed of a first portion formed of the shape memory polymer material and a second portion formed of an alternate shape memory polymer material of a different formulation with a Tg high enough above body temperature that body temperature has no effect on the alternate shape memory polymer material.

In another implementation, a method for treating glaucoma is provided. A shape memory polymer shunt in a predeployed, compressed configuration is implanted substantially through a scleral tunnel either at the limbus or more posterior to the limbus, and into the anterior chamber substantially at the anterior chamber angle whereby a proximal end of the shunt is positioned ab externo and a distal end of the shunt is position ab interno within the anterior chamber. The shape memory polymer shunt is then activated with an external stimulus to transform the shunt into a deployed, expanded configuration that is substantially identical to an original molded configuration. In an additional or alternative embodiment the shunt has a first portion formed of the shape memory polymer material and a second portion formed of an alternate shape memory polymer material of a different formulation with a Tg high enough above body temperature that body temperature has no effect on the alternate shape memory polymer. The second portion is then activated with an alternate external stimulus to transform the second portion into a deployed, expanded configuration that is substantially identical to an original molded configuration.

In a further implementation, a method of manufacturing an aqueous humor bypass shunt is disclosed. A shape memory polymer material is provided with a Tg slightly greater than or equal to human body temperature. The shape memory polymer material is molded into a permanent tubular shunt form. The molded tubular shunt is mechanically compressed radially at a temperature above Tg to deform the molded tubular shunt into a radially smaller form factor. The compressed tubular shunt is then cooled while still in compression to a temperature below Tg to thereby create a stable compressed tubular shunt with a smaller form factor. In an additional embodiment, an alternate shape memory polymer material of a different formulation is provided with a Tg high enough above body temperature that body temperature has no effect on the alternate shape memory polymer material. The molding operation then additionally includes simultaneously and contiguously molding a first portion of the permanent tubular shunt form with the shape memory polymer material and molding a second portion of the permanent tubular shunt form with the alternate shape memory polymer material.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. A more extensive presentation of features, details, utilities, and advantages of the present invention as defined in the claims is provided in the following written description of various embodiments of the invention and illustrated in the accompanying drawings.

### BRIEF DESCRIPTION OF THE DRAWINGS

FIG. 1 is a horizontal cross-section view of an eye.

FIG. 2 is an enlarged view of a portion of the view of FIG. 1 as indicated in FIG. 1.

FIG. 3A is an isometric view of one exemplary implementation of an aqueous humor bypass shunt in a compressed, predeployed configuration.

FIG. 3B is an isometric view of the aqueous humor bypass shunt of FIG. 3A in an expanded, deployed configuration.

FIG. 4 is an isometric view of an alternate exemplary embodiment of an aqueous humor bypass shunt in an expanded, deployed configuration having a circular faceplate for placement ab externo.

FIG. 5 is an isometric view of another exemplary embodiment of an aqueous humor bypass shunt in an expanded, deployed configuration having a trumpet-shaped end for placement ab interno.

FIG. 6 is an isometric view of a further exemplary embodiment of an aqueous humor bypass shunt in an expanded, deployed configuration having multiple lumen for fluid flow.

FIG. 7A is an isometric view of an exemplary embodiment of an aqueous humor bypass shunt in an expanded, deployed configuration having a rectangular faceplate oriented transverse to the axis of the lumen and formed with an distal end of a different polymer formulation than the rest of the shunt as indicated by the stippling.

FIG. 7B is a side elevation view of the aqueous humor bypass shunt of FIG. 7A.

FIG. 7C is a top plan view of the aqueous humor bypass shunt of FIG. 7A.

FIG. 7D is a proximal end elevation view of the aqueous humor bypass shunt of FIG. 7A.

FIG. 7E is a distal end elevation view of the aqueous humor bypass shunt of FIG. 7A.

FIG. 8A is a schematic diagram depicting the insertion of a needle introducer within the eye for placement of the exemplary aqueous humor bypass shunt of FIG. 7A in a compressed, pre-deployed configuration.

FIG. 8B is a schematic diagram depicting the positioning of the aqueous humor bypass shunt of FIG. 7A within the needle introducer using a plunger.

FIG. 8C is a schematic diagram depicting the removal of the needle introducer from about the aqueous humor bypass shunt of FIG. 7A to place the shunt in vivo.

FIG. 8D is a schematic diagram depicting the aqueous humor bypass shunt of FIG. 7A in situ in a compressed, predeployed configuration.

FIG. 8E is a schematic diagram depicting the aqueous humor bypass shunt in situ in a partially deployed, expanded configuration wherein the tip remains compressed until activated for recovery by an additional energy source.

FIG. 8F is a schematic diagram depicting the aqueous humor bypass shunt of FIG. 7A in situ in a fully deployed, expanded configuration.

### DETAILED DESCRIPTION

A small stent, seton, or shunt can be placed within the eye to aide in drainage from the anterior chamber of the eye to the aqueous veins or to secrete from the limbus external to the eye for glaucoma treatment. The terms “stent,” “seton,” and “shunt” are interchangeable within the context of this disclosure. This drainage can decrease the pressure of the eye and potentially modify the course of advancing glaucomatous optic neuropathy as it relates to eye pressure. In the implementations disclosed herein, the shunt is formed of a shape memory polymer material, for example, of the types described below and deformed into a smaller form factor for insertion to reduce trauma to the eye resulting from the procedure. Once in situ, the shunt deploys and expands to its original, larger form factor to provide a secure friction fit of the shunt within the scleral tissue and to enlarge the lumen of the shunt to allow for aqueous flow.

Shape Memory Polymers

Basic thermomechanical response of shape memory polymer (SMP) materials is defined by four critical temperatures. The glass transition temperature, T_{g}, is typically represented by a transition in modulus-temperature space and can be used as a reference point to normalize temperature. SMPs offer the ability to vary T_{g} over a temperature range of several hundred degrees by control of chemistry or structure. The predeformation temperature, T_{d}, is the temperature at which the polymer is deformed into its temporary shape. Depending on the required stress and strain level, the initial deformation at T_{d} can occur above or below T_{g}. The storage temperature, Tₛ, represents the temperature in which no shape recovery occurs and is equal to or below T_{d}. At the recovery temperature, Tᵣ, the shape memory effect is activated, which causes the material to recover its original shape, and is typically in the vicinity of T_{g}. Recovery can be accomplished isothermally by heating to a fixed Tᵣ and then holding, or by continued heating up to and past Tᵣ. From a macroscopic viewpoint, a polymer will demonstrate a useful shape memory effect if it possesses a distinct and significant glass transition and a large difference between the maximum achievable strain, εₘₐₓ, during deformation and permanent plastic strain after recovery, εₚ. The difference εₘₐₓ−εₚ is defined as the recoverable strain, ε_{recover}, while the recovery ratio is defined as ε_{recover}/εₘₐₓ.

The microscopic mechanism responsible for shape memory in polymers depends on both chemistry and structure. The primary driving force for shape recovery in polymers is the low conformational entropy state created and subsequently frozen during the thermomechanical cycle. If the polymer is deformed into its temporary shape at a temperature below T_{g}, or at a temperature where some of the hard polymer regions are below T_{g}, then internal energy restoring forces will also contribute to shape recovery. In either case, to achieve shape memory properties, the polymer must have some degree of chemical crosslinking to form a “memorable” network or must contain a finite fraction of hard regions serving as physical crosslinks.

Shape memory polymer materials may be used for a wide variety of applications. Their ability to recover strains imparted upon them, in a manner that is different than pure thermal expansion, due to an external stimulus, makes SMP materials well suited for many applications, such as biological and general mechanical. The external stimulus that activates SMPs may be heat, light, or other stimuli known to those having skill in the art. SMPs which use heat as an external stimulus often have temperatures at which transition occurs.

A transition temperature can be a property of a material (e.g., SMP, thermoplastic, thermoset). A transition temperature may be defined through a number of methods/measurements and different embodiments may use any of these different methods/measurements. For example, a transition temperature may be defined by a temperature of a material at the onset of a transition (Tₒₙₛₑₜ), the midpoint of a transition, or the completion of a transition. As another example, a transition temperature may be defined by a temperature of a material at which there is a peak in the ratio of a real modulus and an imaginary modulus of a material (e.g., peak tan-δ). It should be noted that the method of measuring the transition temperature of a material may vary, as may the definition of steps taken to measure the transition temperature (e.g., there may be other definitions of tan-δ).

A transition temperature may be related to a number of processes or properties. For example, a transition temperature may relate to a transition from a stiff (e.g., glassy) behavior to a rubbery behavior of a material. As another example, a transition temperature may relate to a melting of soft segments of a material. A transition temperature may be represented by a glass transition temperature (T_{g}), a melting point, or another temperature related to a change in a process in a material or another property of a material.

In addition, molecular and/or microscopic processes, including those processes around a transition temperature, may be related to the macroscopic properties of the material. From a macroscopic viewpoint, as embodied in a modulus-temperature graph, a polymer's shape memory effect may possess a glass transition region, a modulus-temperature plateau in the rubbery state. A polymer's shape memory effect may include, as embodied in a stress-strain graph, a difference between the maximum achievable strain, εₘₐₓ, during deformation and permanent plastic strain after recovery, εₚ. The difference εₘₐₓ−εₚ may be considered the recoverable strain, ε_{recover}, while the recovery ratio (or recovery percentage) may be considered ε_{recover}/εₘₐₓ.

The properties of SMPs can be controlled by changing the formulation of the SMP, or by changing the treatment of the SMP through polymerization and/or handling after polymerization. The techniques of controlling SMP properties rely on an understanding of how SMP properties are affected by these changes and how some of these changes may affect more than one property. For example, changing the percentage weight of a cross-linker in a SMP formulation may change both a transition temperature of the SMP and a modulus of the SMP. In one embodiment, changing the percentage weight of a cross-linker will affect the glass transition temperature and the rubbery modulus of an SMP. In another embodiment, changing the percentage weight of cross-linker will affect a recovery time characteristic of the SMP.

Some properties of a SMP may be interrelated such that controlling one property has a strong or determinative effect on another property, given certain assumed parameters. For example, the force exerted by a SMP against a constraint after the SMP has been activated may be changed through control of the rubbery modulus of the SMP. Several factors, including a level of residual strain in the SMP enforced by the constraint, will dictate the stress applied by the SMP, based on the modulus of the SMP. The stress applied by the SMP is related to the force exerted on the constraint by known relationships.

Examples of constituent parts of the SMP formulation include monomers, multi-functional monomers, cross-linkers, initiators (e.g., photo-initiators), and dissolving materials (e.g., drugs, salts). Two commonly included constituent parts are a linear chain and a cross-linker, each of which are common organic compounds such as monomers, multi-functional monomers, and polymers.

A cross-linker (or “crosslinker”), as used herein, may mean any compound comprising two or more functional groups (e.g., acrylate, methacrylate), such as any poly-functional monomer. For example, a multi-functional monomer is a poly ethylene glycol (PEG) molecule comprising at least two functional groups, such as di-methacrylate (DMA), or the combined molecule of PEGDMA. The percentage weight of cross-linker indicates the amount of the poly-functional monomers placed in the mixture prior to polymerization (e.g., as a function of weight), and not necessarily any direct physical indication of the as-polymerized “crosslink density.”

Because SMP material requires both a thermal transition and a form of crosslinking to possess shape-memory characteristics, the polymer is typically synthesized from a linear chain building mono-functional monomer (tert-butyl acrylate) and a crosslinking di-functional monomer (poly (ethylene glycol) dimethacrylate). Because the crosslinking monomer has two methacrylate groups, one at each end, it is possible to connect the linear chains together. This linear monomer portion can be used to help control the glass transition temperature of the network as well as its overall tendency to interact with water. Thus, the linear portion of the network remains an important and tailor-able portion of the composition.

A linear chain may be selected based on a requirement of a particular application, because of the ranges of rubbery moduli and recovery forces achieved by various compositions. In one embodiment, a SMP with a high recovery force and rubbery modulus may be made from a formulation with methyl-methacrylate (MMA) as the linear chain. In another embodiment, a SMP with a lower recovery force and rubbery modulus may be made from a formulation with tert-butyl acrylate (tBA) as the linear chain. In other embodiments, other linear chains may be selected based on desired properties such as recovery force and rubbery modulus.

In one embodiment, the copolymer network consists of two acrylate-based monomers. In one example of this embodiment, tert-butyl acrylate may be crosslinked with poly (ethylene glycol), dimethacrylate (PEGDMA) via photopolymerization to form a cross-linked network. One subset of this formulation may consist of 10 wt % PEGDMA with a Mₙ=1000 and remainder tert-butyl acrylate with 0.1 wt % photoinitiator (2,2 dimethoxy-2-phenylacetopenone). This exemplary polymer network has a glass transition temperature T_{g} of about 45° C., which offers shape memory activation along with a reasonably soft compliance at body temperature. Furthermore, it has a low rubbery modulus of approximately 1-2 MPa, which is indicative of a low degree of crosslinking that allows for greater packaging deformations and higher strains to failure. In some embodiments, the molecular weight of the PEGDMA may be varied to control hydrophobicity and/or hydrophylicity. This may allow better integration with medical fabrics or meshes that are generally more hydrophobic or hydrophilic. In some embodiments, where a stiffer medical fabric is used, the PEGDMA:tBA wt percentage is increased such that the SMP has sufficient stored force to allow deployment of the SMP integrated material. In still other embodiments, addition of thiol groups may allow better control of manufacturing for composite systems since oxygen inhibition could be decreased leading to better polymerization.

The SMP material may be further varied to enhance desired properties. The SMP material may be photopolymerized from several different monomers and/or homopolymers to achieve a range of desired thermomechanical properties. A SMP formed from three or more monomers and/or homopolymers may achieve a range of rubbery modulus to glass transition temperatures, rather than a strictly linear relationship between these two thermomechanical properties. For example, tert-butyl acrylate may be substituted by 2-hydroxyethyl methacrylate or methyl methylacrylate to create either more hydrophilic or stronger networks, if desired. Additionally, if a hydrophilic monomer such as 2-hydroxyethyl methacrylate is substituted for tert-butyl acrylate, the SMP has the ability to swell post-implantation through hydrogel mechanisms.

Representative natural polymer blocks or polymers include proteins such as zein, modified zein, casein, gelatin, gluten, serum albumin, and collagen, and polysaccharides such as alginate, celluloses, dextrans, pullulane, and polyhyaluronic acid, as well as chitin, poly(3-hydroxyalkanoate)s, especially poly(.beta.-hydroxybutyrate), poly(3-hydroxyoctanoate) and poly(3-hydroxy fatty acids). Representative natural biodegradable polymer blocks or polymers include polysaccharides such as alginate, dextran, cellulose, collagen, and chemical derivatives thereof (substitutions, additions of chemical groups, for example, alkyl, alkylene, hydroxylations, oxidations, and other modifications routinely made by those skilled in the art), and proteins such as albumin, zein and copolymers and blends thereof, alone or in combination with synthetic polymers.

Representative synthetic polymer blocks or polymers include polyphosphazenes, poly(vinyl alcohols), polyamides, polyester amides, poly(amino acid)s, synthetic poly(amino acids), polyanhydrides, polycarbonates, polyacrylates, polyalkylenes, polyacrylamides, polyalkylene glycols, polyalkylene oxides, polyalkylene terephthalates, polyortho esters, polyvinyl ethers, polyvinyl esters, polyvinyl halides, polyvinylpyrrolidone, polyesters, polylactides, polyglycolides, polysiloxanes, polyurethanes and copolymers thereof. Examples of polyacrylates include poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate) and poly(octadecyl acrylate).

Synthetically modified natural polymers include cellulose derivatives such as alkyl celluloses, hydroxyalkyl celluloses, cellulose ethers, cellulose esters, nitrocelluloses, and chitosan. Examples of cellulose derivatives include methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate and cellulose sulfate sodium salt. These are collectively referred to herein as “celluloses”.

Representative synthetic degradable polymer segments include polyhydroxy acids, such as polylactides, polyglycolides and copolymers thereof; poly(ethylene terephthalate); polyanhydrides, poly(hydroxybutyric acid); poly(hydroxyvaleric acid); poly[lactide-co-(.epsilon.-caprolactone)]; poly[glycolide-co-(.epsilon.-caprolactone)]; polycarbonates, poly(pseudo amino acids); poly(amino acids); poly(hydroxyalkanoate)s; polyanhydrides; polyortho esters; and blends and copolymers thereof. Polymers containing labile bonds, such as polyanhydrides and polyesters, are well known for their hydrolytic reactivity. Hydrolytic degradation rates of these polymers may be altered by simple changes in the polymer backbone and the polymer's sequence structure.

Examples of non-biodegradable synthetic polymer segments include ethylene vinyl acetate, poly(meth)acrylic acid, polyamides, polyethylene, polypropylene, polystyrene, polyvinyl chloride, polyvinylphenol, and copolymers and mixtures thereof.

Hydrogels can be formed from polyethylene glycol, polyethylene oxide, polyvinyl alcohol, polyvinyl pyrrolidone, polyacrylates, poly (ethylene terephthalate), poly(vinyl acetate), and copolymers and blends thereof.

The polymers can be obtained from commercial sources such as Sigma Chemical Co., St. Louis, Mo.; Polysciences, Warrenton, Pa.; Aldrich Chemical Co., Milwaukee, Wis.; Fluka, Ronkonkoma, N.Y.; and BioRad, Richmond, Calif. Alternately, the polymers can be synthesized from monomers obtained from commercial sources.

Various SMP properties may be controlled via variations in a cross-linker in the SMP formulation. A range of average molecular weights of cross-linker material for use in a SMP may be determined based upon the desired transition temperature, for example, a transition temperature close to human body temperature. The transition temperature affects the range of possible average molecular weights of cross-linker material that may be used in the SMP because certain combinations of average molecular weights and of percentage weights of cross-linker produce certain transition temperatures and other combinations produce other transition temperatures.

A range of percentage weights of cross-linker material for use in a SMP is also determined from the selected transition temperature. Certain combinations of average molecular weights of cross-linker and percentage weights of cross-linker may be used in the SMP formulation to achieve a certain transition temperature. Determining the range of percentage weight cross-linker and the range of molecular weights may be performed based upon a relationship between transition temperature, molecular weight, and percentage weight cross-linker. The relationship is specific to the linear chain and cross-linker used. Other inputs or manufacturing techniques may also affect the relationship and eventual transition temperature of a SMP.

In one embodiment, empirically-derived relationships which relate molecular weight and weight percentage cross-linker to (a) the transition temperature, (b) the rubbery modulus, and/or (c) a recovery time characteristic may be used. The range of rubbery moduli is determined by evaluating the relationship between rubbery modulus, percentage weight of cross-linker, and molecular weights for a number of combinations determined. This results in a range of possible rubbery moduli for SMPs that also has the desired transition temperature. In another embodiment, relationships may be derived from known theoretical models.

A rubbery modulus is selected from a range of rubbery moduli of as an initial goal value of rubbery modulus for the SMP. The modulus selection may alternatively be performed after a transition temperature is selected, which produces another range of rubbery moduli. In other words, the method may be performed iteratively, repeatedly, and/or in parts. The molecular weight and percentage weight of cross-linker is determined based on the selected rubbery modulus by using the relationship between rubbery modulus, molecular weight and percentage weight of cross-linker to find the combination of molecular weight and percentage weight that corresponds to the rubbery modulus selected.

In another embodiment, determining a range of molecular weights and percentage weights of cross-linker may be performed by creating and/or selecting a table, graph, or chart corresponding to a desired transition temperature or a desired rubbery modulus among a plurality of tables, graphs, and/or charts. In this embodiment, the tables, graphs, and/or charts include information from the relationships described above and outline ranges of molecular weights and percentage weights cross-linker that correspond to the desired value of the property (e.g., transition temperature).

In some embodiments, the SMP may be created to elute various drugs through the incorporation of these drugs into the matrix of the SMP, through addition of other materials such as poly(lactic-co-glycolic acid) (PLGA) with an embedded drug onto the surface of the SMP, by holding the drug within a cavity or lumen of a device molded or machined from the SMP, or through surface coating of the drug onto the SMP.

In some implementations, the shape memory polymer may comprise thiol and/or vinyl monomers or oligomers. In some implementations, monomers or oligomers with acrylate or methacrylate functional groups may be combined with thiol and/or vinyl monomers or oligomers. Thiol groups may be added through chain transfer processes.

A thiol-vinyl SMP system includes molecules containing one or more thiol functional groups, which terminate with —SH, and molecules containing one or more vinyl functional groups, which contain one or more carbon-carbon double bonds. The vinyl functional groups in the system may be provided by, for example, allyl ethers, vinyl ethers, norborenes, acrylates, methacrylates, acrylamides or other monomers containing vinyl groups. In some implementations, additional fillers, molecules, and functional groups may be provided to tailor and provide additional properties. In different embodiments, the thiol-ene system has about 1-90% of its functional groups as thiol functional groups or 2%-65% thiol functional groups. The balance of the functional groups (35% to 98% of the functional groups may be vinyl functional groups. In an embodiment, 5-60 mol % of the functional groups in the system may be thiol functional groups and 95-40 mol % vinyl functional groups. In the present invention, the system of molecules containing thiol functional groups and the molecules forming vinyl functional groups is capable of forming a network.

In one class of thiol-vinyl systems, the vinyl monomer is not readily homopolymerizable and is termed an ene monomer. In these systems, the polymerization proceeds via a radically initiated step growth reaction between multifunctional thiol and ene monomers. The reaction proceeds sequentially, via propagation of a thiyl radical through a vinyl functional group. This reaction is followed by a chain transfer of a hydrogen radical from the thiol which regenerates the thiyl radical. the process then cycles many times for each radical generated in the photoinitiation step. This successive propagation/chain transfer mechanism is the basis for thiol-ene polymerization.

Thiol bearing monomers suitable for implementations of thiol-vinyl shape memory polymer systems include any monomer or oligomer having thiol (mercaptan or “SH”) functional groups. Thiols are any of various organic compounds or inorganic compounds having the general formula RSH which are analogous to alcohols but in which sulfur replaces the oxygen of the hydroxyl group. Suitable monomers or oligomers may have one or more functional thiol groups. In an embodiment, the monomer or oligomer cannot be considered a polymer in its own right. In different embodiments, the monomer or oligomer has an average molecular weight less than 10,000, less than 5,000, less than 2,500, less than 1000, less than 500, from 200 to 500, from 200-1000, from 200-1,500, from 200-2000, from 200-2,500, from 200-5000, or from 200-10,000. In different embodiments, the monomer or oligomer has at least two thiol functional groups, at least three thiol functional groups, at least four thiol functional groups, at least five thiol functional groups, at least six thiol functional groups or from 2 to 4 thiol functional groups. Examples of suitable thiol bearing monomers include: pentaerythritol tetra(3-mercaptopropionate) (PETMP); trimethylolpropane tris(3-mercaptopropionate) (TMPTMP); glycol dimercaptopropionate (GDMP); IPDU6Th; and 1,6-hexanedithiol (HDTT), and benzene diol.

Monomers or oligomers having vinyl functional groups suitable for implementations of thiol-vinyl shape memory polymer systems include any monomer or oligomer having one or more functional vinyl groups, i.e., reaching “C═C” groups. In an embodiment, the monomer or oligomer cannot be considered a polymer in its own right. In different embodiments, the monomer or oligomer has an average molecular weight less than 10,000, less than 5,000, less than 2,500, less than 1000, less than 500, from 200 to 500, from 200-1000, from 200-1,500, from 200-2000, from 200-2,500, from 200-5000, or from 200-10,000. In different embodiments, the monomer or oligomer has at least two vinyl functional groups, at least three vinyl functional groups, at least four vinyl functional groups, at least five vinyl functional groups, at least six vinyl functional groups, or from 2 to 4 vinyl functional groups. Examples of suitable vinyl monomers include: allyl pentaerythritol (APE); Wallyl triazine trione (TATATO); trimethylolopropane diallyl ether (TMPDAE); hexanediol diacrylate (HDDA); trimethylolpropane triacrylate (TMPA); Ebecryl 8402; Vectomer 5015; and IPDU6AE.

Monomers or oligomers with acrylate or methacrylate functional groups may also be combined with thiol and/or vinyl monomers or oligomers using, as one example, a chain transfer agent process with the agent being thiol. Exemplary acrylate and methacrylate monomers for use with thiol-vinyl shape memory polymer systems include tricyclodecane dimethanol diacrylate; tricyclodecane dimethanol dimethacrylate; bisphenol-A ethoxylated diacrylate; bisphenol-A ethoxylated dimethacrylate; bisphenol-A epoxy diacrylate; bisphenol-A epoxy dimethacrylate; urethane acrylates; urethane methacrylates; polyethylene glycol diacrylate; polyethylene glycol dimethacrylate and commercial monomers. Commercial monomers include aliphatic urethane acrylates such as Ebecryl 8402; Ebecryl 230; Loctite 3494; Ebecryl 4833; Ebecryl 3708.

The monomer or oligomer comprising a vinyl group may further comprise at least one urethane group. In an embodiment, the monomer comprises from 2-4 or 2-6 urethane groups. In an embodiment, the oligomer comprises from 4-40 urethane groups. A monomer comprising urethane groups may be formed by reacting a polyisocyanate with a molecule comprising an alcohol group and at least two vinyl groups. For example, a diisocyanate could be reacted with a trimethylolpropane diallyl ether or allyl pentaerythritol.

Thiol-vinyl systems for shape memory polymers may also include and/or utilize various initiators, fillers, and accelerators, depending on the application. For example, if photopolymerization using visible light is desired, a commercially available photoinitiator such as Irgacure 819 or Irgacure 784 (manufactured by Ciba Specialty Chemicals Co. (http://www.cibasc.com)) may be used. If ultraviolet photopolymerization is desired, then 2,2-dimethyloxy-2-pheynlacetophenone (Irgacure 651, Ciba Specialty Chemicals Co.) may be used as an initiator or 1-hydroxy-cyclohexyl-phenyl-ketone (Irgacure 184, Ciba Specialty Chemicals).

A thiol-yne system includes molecules containing one or more thiol functional groups, which terminate with —SH, and molecules containing one or more yne functional groups, which contain one or more carbon-carbon triple bonds. The functional groups in the system may be provided by, octadiyne or heptadiyne for example, or other monomers containing yne groups.

Ocular Anatomy

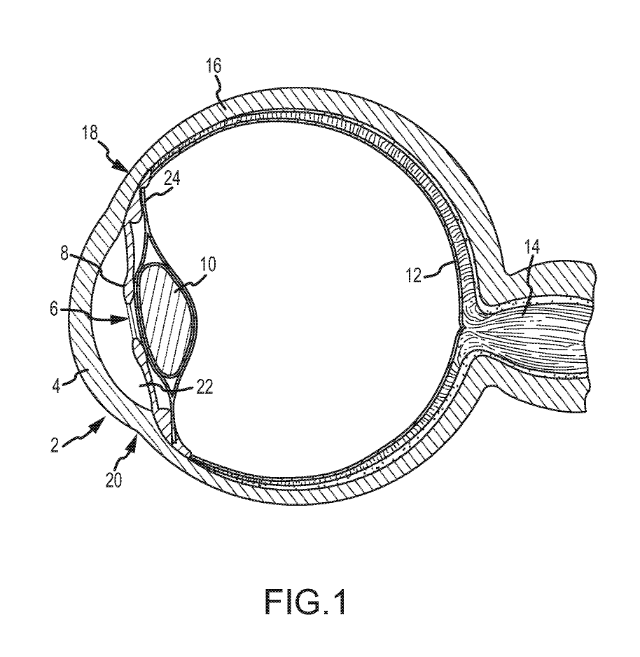

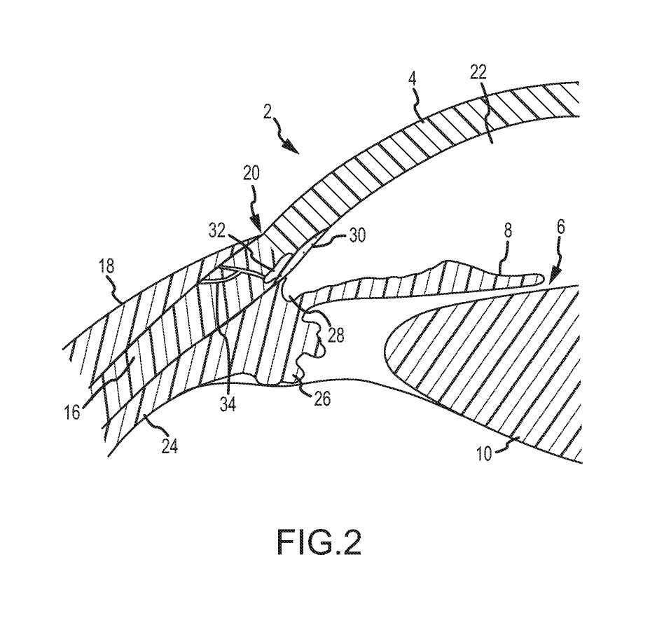

For background illustration purposes, FIG. 1 is a horizontal sectional view of an eye 2 and FIG. 2 is an enlarged view of a portion of the eye shown in FIG. 1. The cornea 4, pupil 6, iris 8, lens 10, retina 12, and optic nerve 14 are all depicted in FIG. 1. A thick collagenous tissue known as sclera 16 (the white tissue) covers the entire eye 2 except that portion covered by the cornea 4. The sclera 16 is further covered by a clear mucous membrane called the conjunctive 18 that also terminates at the cornea 4. The cornea 4 merges into the sclera 16 at a juncture referred to as the limbus 20. The cornea 4 is a thin transparent tissue that focuses and transmits light into the eye 2 through the pupil 6, which is the circular hole in the center of the iris 8 (the colored portion of the eye). The light passes through the pupil 6 and is further focused by the lens 10 and then travels to the retina 12. The optic nerve 14 transmits visual information recorded in the retina 12 to the brain.

The void between the cornea 4 and the lens 10 is called the anterior chamber 22 and is filled with a clear fluid called aqueous humor. The aqueous humor maintains intraocular pressure and inflates the globe of the eye. The optic nerve 14 can be damaged over time by excessive intraocular pressure, which is the pathology known as glaucoma.

FIG. 2 shows an enlarged view of the area around the limbus 20 including the relative anatomical locations of the anterior chamber 22, the iris 8, the ciliary processes 26, the trabecular meshwork 30, and Schlemm's canal 32. The ciliary processes 26 begins internally in the eye 2 and extends along the interior of the sclera 16 and becomes the choroid 24. The choroid 24 is a vascular layer of the eye 2 underlying retina 18. The iris 8 extends from the ciliary processes 26 in a opposite direction, radially into the anterior chamber 22. Aqueous humor is produced primarily by the ciliary processes 26 and reaches the anterior chamber angle 28 formed between the iris 8 and the cornea 4 through the pupil 6. In a normal eye, the aqueous humor is removed through the trabecular meshwork 30 formed in the anterior chamber angle 28.

Aqueous humor passes through trabecular meshwork 30 into Schlemm's canal 32 and through the aqueous veins 34 which merge with blood-carrying veins and into venous circulation. Intraocular pressure of the eye 2 is maintained by the intricate balance of secretion of aqueous humor from the ciliary processes 26 and outflow of the aqueous humor via the trabeclular meshwork 30, Schlemm's canal 32 and the aqueous veins 34 in the manner described above. An imbalance between aqueous humor secretion and outflow may result in either an excess or a dearth of aqueous humor in the anterior chamber 22. Glaucoma is characterized by the excessive buildup of aqueous fluid in the anterior chamber 22, which produces an increase in intraocular pressure because fluids are relatively incompressible and pressure is translated to other areas of the eye.

It may be appreciated that one measure for addressing increased pressure in the anterior chamber 22 is to aid in the removal of aqueous humor that is not being adequately removed by the natural systems. One methodology that has shown some measure of success is to implant a shunt device through the sclera 16, through Schlemm's canal 32, and through the trabecular meshwork 30 directly into the anterior chamber angle 28 in order to transport aqueous humor directly from the anterior chamber 22 to a pocket between the conjunctiva 18 and the sclera 16. The shunt may augment or bypass the natural physiological outflow pathways of the trabecular meshwork 30 and Schlemm's canal 34 to transport the aqueous humor to the aqueous vein collectors 34 to help balance the intraocular pressure.

Shape Memory Polymer Shunts

Tailoring of specific SMP formulations allows shunts to be created to meet specific design requirements and to be manufactured using scalable liquid injection manufacturing techniques. SMP formulations were developed to optimize the following properties:
Shape fixity of >98.5% (defined as the percent change in recovered shape compared to the original molded shape);
Recovery rates of between 0.25 seconds to 600 seconds;
Minimum device deformations of at least 40% in any dimension during the manufacturing process, and preferentially of 100-200%;
Rubbery modulus of 250 kPa to 20,000 kPa
Coloration of blue, yellow, red and green, or combinations thereof
Cycle times for liquid injection manufacturing of 30 seconds to 20 minutes
Ability to tolerate high temperature mold-based manufacturing, e.g., temperatures of as much as 400 degrees
Capability to tolerate high-pressure mold-based manufacturing, specifically pressures of as much as 50 Mpa;
Ability to flow through extremely narrow channels (<100 microns diameter) during the mold-based manufacturing process (i.e., low viscosity at manufacturing temperatures); and
Volume shrinkage to permanent shape of less than 3% after thermal curing in the mold-based manufacturing process.
   Some exemplary SMP formulations and their measured properties are reported in Table A below. In one formulation, tert-butyl acrylate (tBA) is combined with poly(ethylene glycol) dimethacrylate (PEGDMA) 550 as a cross-linker. The weight percentages of each may be varied to design an SMP with particular desired material properties.

TABLE A

| | | | | Recovery | | Injection | | Max | Minimum | Maximum |
|---|---|---|---|---|---|---|---|---|---|---|
| | | Rubbery | | time at | | cycle | | Injection | Gate | Exposure |
| | Tg | Modulus | Maximum | 37 C. | | times | Volume | Pressure | Dimension | Temp |
| Formulations | (° C.) | (MPa) | Strain | (sec) | Color | (mins) | Shrinkage | (MPa) | (microns) | (° C.) |
| 80% tBA- | 52-55 | 5 | 100% | 300 | Blue | 0.5-60 | <1% | 55 | 10 | 250 |
| 20% PEGDMA | | | | | red | | | | | |
| 550 | | | | | yellow | | | | | |
| | | | | | green | | | | | |
| 70% tBA- | 40 | 5 | 100% | 10 | Blue | 0.5-60 | <1% | 55 | 10 | 250 |
| 20% PEGDMA | | | | | red | | | | | |
| 550-10% nBA | | | | | yellow | | | | | |
| | | | | | green | | | | | |
| tBA or n-BA | −36-114 | 1 | 180% | 1-600 | Blue | 0.5-60 | <1% | 55 | 10 | 250 |
| (or combination | | | | | red | | | | | |
| with 5% | | | | | yellow | | | | | |
| Crosslinking | | | | | green | | | | | |
| tBA or n-BA | −30-96 | 5 | 100% | 0.5-400 | Blue | 0.5-60 | <1% | 55 | 10 | 250 |
| (or combination) | | | | | red | | | | | |
| with 20% | | | | | yellow | | | | | |
| Crosslinking | | | | | green | | | | | |
| tBA or n-BA | −21-73 | 14 | 60% | 0.25-300 | Blue | 0.5-60 | <1% | 55 | 10 | 250 |
| (or combination) | | | | | red | | | | | |
| with 40% | | | | | yellow | | | | | |
| Crosslinking | | | | | green | | | | | |

As one example of the optimization, recovery time is controlled by the relationship of the glass transition temperature (Tg) of the SMP material used to the environmental temperature (Te) an SMP device is deployed in. A Tg<Te deploys more slowly than a Tg=Te, and a Tg>Te deploys at the fastest rate. Tg of the material may be controlled from −35° C. up to 114° C. allowing a wide range of control over the deployment rate into the body. Devices have been created that deploy in less than a second all the way up to several minutes to fully deploy. In some embodiments, a the SMP may have a transition temperature (Tr) that is tailored to allow recovery at the body temperature, Tᵣ˜T_{g}˜37° C. With such an SMP formulation, the thermal energy of the patient may be used to activate the SMP shunt.

In order to deliver the stent through the smallest possible incision, the mechanical properties of the SMP devices may be developed to achieve high levels of recoverable strain. In tension, up to 180% strain can be achieved for 10% cross-linked systems and up to 60% strain can be achieved in 40% cross-linked systems. In compression 80% or more strain can be achieved with the above percentage cross-link. The desired levels of strain in tension and compression are determined by the level of deformation required to fit the SMP stent into the delivery system. Formulations with lower amounts of cross-linking can undergo higher levels of deformation without failure. Exemplary SMP stent embodiments utilize 5%-40% cross-linking to achieve the material properties for the desired level of recoverable strain.

Manufacturing of SMP stents may be achieved through either thermal initiation or photo-initiation or a combination of the two processes. For thermal initiation, both peroxides and azo initiators have been utilized. 2,2-dimethoxy-2-phenylacetophenone (DMPA) may be used for photo-initiation. Formulations vary in quantity from 0.01% by weight to 1% by weight of initiator. These are varied to optimize cycle time during the manufacturing process and still maintain desired thermomechanical properties.

Colorant can also be added to the formulations. SMP materials with SPECTRAFLO (trademark of Ferro) liquid colors have been created. Formulations with 0.1% to 2% by weight have been created, which allows various colors to be added yet maintain desired thermomechanical properties.

The post deployment shape should be highly controlled to maximize the efficacy and control the deployment time of the shunt. The higher the shape fixity, the higher the reproducibility and confidence that the deployed shunt will function as intended. The SMP materials disclosed herein provide extremely high shape fixity (>95-99%). This is in large part because the SMP materials deploy using a non-elastic, non-melt shape recovery process (i.e., it is not a phase change using fluid properties). Further, the SMP materials are not a hydrogel or other type of hydrating material. The SMP materials transform from one highly-reproducible, non-changing, non-creeping, non-deforming, storage shape, to another highly-reproducible, non-changing, non-creeping, non-deforming, secondary (permanent) shape.

The SMP materials have a pre-programmed shape; post-deployment the SMP devices release internal stored energy to move to the programmed shape, which may or may not be adaptive to the local tissue. The local tissue does not play a part in shaping the form of the SMP devices. The SMP devices return to their “permanent” shape as originally formed when molded, before being deformed for smaller profile delivery. The speed of full deployment from the deformed state to the glass (permanent) state can be varied over a wide range from less than a second to over 600 seconds depending upon the SMP formulation.

Additionally, because of the high Tg (i.e., at or above body temperature) of the SMP formulations, the processes of packaging, shipping, storing, and ultimately implanting SMP devices does not require refrigerated storage or ice or an otherwise low-temperature operating environment. Thus, a significant advantage of the SMP materials described herein is that they can be stored in the stored shape for extended periods of time, they can be packaged in constrained forms within a customized delivery system, and they can be deployed without need for prior refrigeration or other temperature changes.

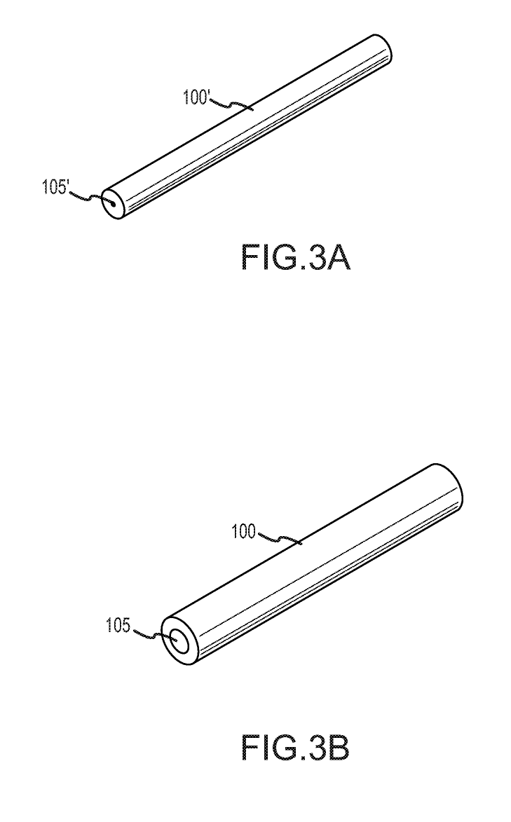

FIG. 3A shows an exemplary implementation of a shunt 100′ in a deformed, predeployed state. FIG. 3B depicts the same shunt 100 of FIG. 3A in its original and post-deployed shape. In this exemplary embodiment, the shunt 100 is formed as a tube defining a lumen 105. The lumen 105 may extend from a proximal end to a distal end of the shunt 100. As indicated in FIG. 3A, the shunt 100′ is compressed radially with respect to a longitudinal axis of the lumen 105′ and the lumen 105′ is collapsed as compared to the form of the shunt 100 in the deployed state of FIG. 3B wherein the shunt 100 is expanded radially and the lumen 105 is open and of a larger diameter. It should be noted that various other shapes and sizes of shunts may be used. For example, the shunt 100 may have a cross-sectional form of any geometry besides the circular cross section shown in FIGS. 3A and 3B. Further, the deformed configuration of the predeployed shunt 100′ may take any form that is advantageous for implantation that could aid in minimizing the trauma to the eye 2 during implantation.

The shunt 100 may be sized longitudinally to span the distance between the anterior chamber and the scleral surface at the limbus 20. Typically, as described further below, a flap of sclera 16 and conjunctiva 18 or conjunctiva 18 alone will cover the proximal end of the shunt 100 after implantation. In exemplary embodiments, the shunt 100 may have a length between about 1 and 10 millimeters in a deployed configuration. In exemplary embodiments, the shunt 100 may have an outside diameter between about 50 and 800 microns in a deployed configuration. In exemplary embodiments, the lumen 105 may have an inside diameter between about 50 and 500 microns in a deployed configuration. Other diameters are possible and contemplated based upon the particular application or need. The shunt 100 may be made in a variety of lengths and diameters for “off-the-shelf” selection to accommodate varying individual patient physiologies.

As indicated, the shunt 100 may be formed of a shape memory polymer material as described above. For example, any of the shape memory polymers described herein can be made with any of the properties described in U.S. patent application Ser. No. 12/520,399, filed Apr. 20, 2010, or in U.S. patent application Ser. No. 12/988,983, filed 5 Jan. 2011 entitled “Thiol-vinyl and thiol-yne systems for shape memory polymers,” previously referenced above. In some embodiments the shunt 100 may be made from a combination of different shape memory polymer formulations used to form various portions of the shunt 100 as will be further described below.

In one exemplary implementation, the SMP shunt 100 may be formed by injection molding or machining processes (e.g., lathing, or cryolathing) one of the formulations described above. To create a shunt 100, a combination of machining processes such as lathing, milling, boring/drilling, and abrasive machining may all be employed to create a finished device. In an exemplary embodiment an 80-20 (tBA-PEGDMA 550) combination may be used. This tBA-PEGDMA 550 mixture has extremely low viscosity when heated in the mold and is thus able to easily flow through and fill the mold to form the very small diameter lumen 105. Once cooled and released from the mold, the SMP shunt 100 is in its permanent form. However, for implantation, it is desirable to reduce the size and form factor of the SMP shunt 100 such that it can be implanted through a smaller incision than typical shunts of this type.

The molded SMP shunt 100 may next be placed within a fabric sheath or sock for transmission of the SMP shunt 100 through a compression die. The fabric sock may be closed at one end and open at an opposite end and sized to fit snugly around the SMP shunt 100. The fabric sock may be significantly longer than the length of the SMP shunt 100 in order to assist in pulling the SMP shunt 100 through a compression die. In an exemplary implementation, the fabric sock may be made of a silk fabric.

The compression die may define a borehole extending laterally therethrough from an entrance side to and exit side. The borehole in the compression die may be divided into several sections of varying diameter. An entrance section opening up to the entrance side may be of a constant diameter of slightly larger than the diameter of the SMP shunt 100 such that the SMP shunt 100 can be easily inserted into the borehole of the compression die. A middle section of the borehole may taper in diameter from the diameter of the entrance section to a smaller diameter that transitions into and is congruent with a diameter of an exit section that opens the exit side. The diameter of the exit section may be congruent with a desired final diameter of the SMP shunt 100′ in a compressed, predeployed configuration.

The open end of the fabric sock may be placed within the borehole from the entrance side and is long enough to extend the length of the borehole and extend out of the exit side. The open end of the fabric sock may then be grasped to pull the SMP shunt 100 within the fabric sock into the entrance section of the borehole. The compression die may then be heated to a temperature greater than Tg for the SMP formulation used until the SMP shunt 100 reaches a temperature greater than Tg and is softened. The fabric sock is then pulled through the borehole whereby the SMP shunt 100 is likewise pulled through the middle section and radially compressed. The compressed SMP shunt 100′ is then left in the reduced diameter exit section while the compression die and the compressed SMP shunt 100′ therein are cooled to a temperature below Tg, thereby locking the compressed SMP shunt 100′ in the compressed state. Once the compressed SMP shunt 100′ has been cooled below Tg, it can be removed from the compression die and the fabric sock and it will remain in the compressed shape with a smaller than original diameter for packaging, storage, and ultimately implantation as further described below.

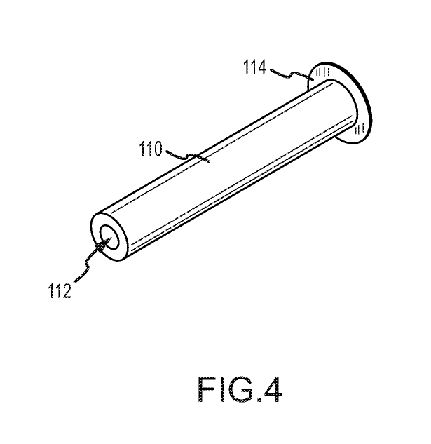

An alternative exemplary embodiment of a SMP shunt 110 in a deployed/expanded configuration is shown in FIG. 4. The shunt 110 of FIG. 4 is similar to the shunt 100 of FIG. 3B as it is formed as a cylindrical tube defining a lumen 112. In addition, the shunt 110 has a circular faceplate 114 extending as a flange from the proximal end of the shunt 110. While a circular faceplate 114 is shown, any shape (e.g., polygonal, oval, amorphous) of faceplate can be used. Moreover, while the faceplate 114 of the shunt 110 is shown as being formed in a plane normal to the longitudinal axis of the shunt 110, an faceplate may be coupled with shunt at any number of interface angles. The faceplate 114 may be provided to help anchor the shunt 110 external to the eye 2 and prevent it from migrating into the anterior chamber 22. In some embodiments, the faceplates 114 may have a diameter or other cross-sectional dimension ranging between half a millimeter and three millimeters. A faceplate can also be used to create a pocket within the sclera 16 for fluid to flow into as further described below.

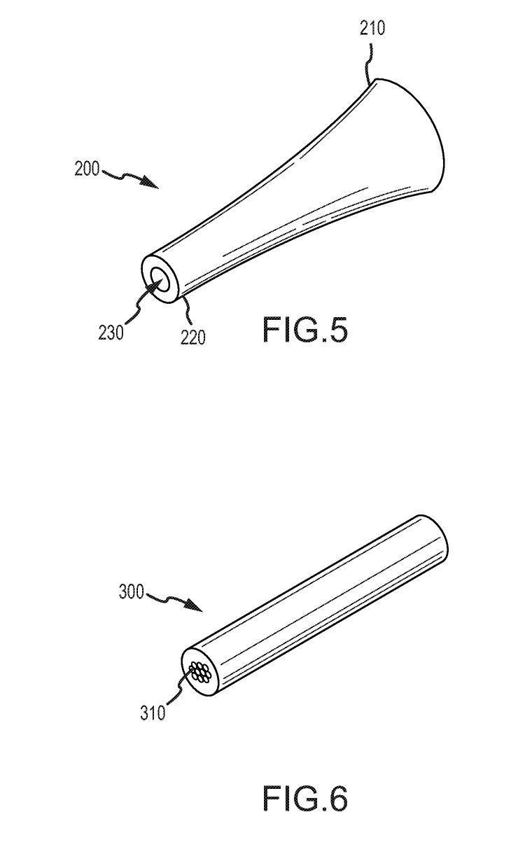

FIG. 5 depicts another embodiment of an SMP shunt 200 in a deployed/expanded configuration that is “funnel-shaped”, i.e., the outer diameter tapers from a distal end 210 to a proximal end 220. The shunt 200 defines a lumen 230 that extends from the proximal end 220 to the distal end 210. The shunt 200 has an outer diameter or cross section at the distal end 210 that is larger than the diameter or cross section at the proximal end 220. The lumen 230 of the shunt 200 may similarly be larger in diameter at the distal end 210 than at the proximal end 220. With this configuration, the shunt 200 may provide a larger opening into the anterior chamber 22 to create a greater basin for collection of aqueous humor for transport out of the anterior chamber 22.

FIG. 6 depicts a further exemplary embodiment of a SMP shunt 300 that defines a plurality of lumens 310 extending therethrough. The various lumens 310 can be grouped together (as shown) or separated throughout the body of the shunt 300. By providing a plurality of lumen 310, the shunt 300 can be titrated by slowly opening one or more lumen with an external activation source (e.g., a laser). The lumens 310 may initially be plugged or collapsed and then selectively unplugged or expanded through external activation. In this respect, the plugs or a portion of the wall of the shunt 300 forming the lumens 310 may be of a different SMP formulation (e.g., with a higher Tg) than the rest of the shunt 300 that is activated by body heat. The lumen may later be opened individually by a separate activation method (e.g., exposure to a higher temperature. Each lumen 310 may also hold a different medication and opening each separately can deliver a specific medication at a specific time. The lumens 310 may be of different sizes and can therefore provide greater or lesser pressure relief based upon the lumen size and its ability to port the aqueous humor from the anterior chamber 22. Different lumens 310 may also lead to different areas of drainage. For example, if one lumen is initially opened and the adjacent tissue under the scleral flap 52 scars, another lumen unaffected by the scar tissue and draining to a different location may be opened to provide additional pressure relief. Any of the shunts described in this disclosure can include a plurality of lumens like shunt 300.

Further, in alternate exemplary implementations, one or more of the lumens 310 my be fitted with a one-way valve. The valves may be fitted either within the lumen or at the surface of the faceplate (if part of the particular shunt design) to allow for flow of aqueous out of, but not into, the eye. In one implementation, a valve may be formed of SMP material in a separate molding operation and then inserted into the lumen of the shunt before the shut is deformed and compressed into its predeployment form. In another implementation, the valve may be molded directly into the lumen of the shunt using a modified pin mold. Such one-way valves may also be formed with filters to retard migration of bacteria through the shunt into the eye. In one exemplary embodiment the valves may be designed with specific bias forces such that they act to gauge pressure and only open at certain pressures (e.g., if the intraocular pressure is too high). A biologic filter with micropores ranging from 1-20μ may be formed directly within the lumen of the shunt through a modified thermal molding process, or they may be provided as part of the one-way valve structure through a separate molding process for insertion into the lumen as part of the valve device.

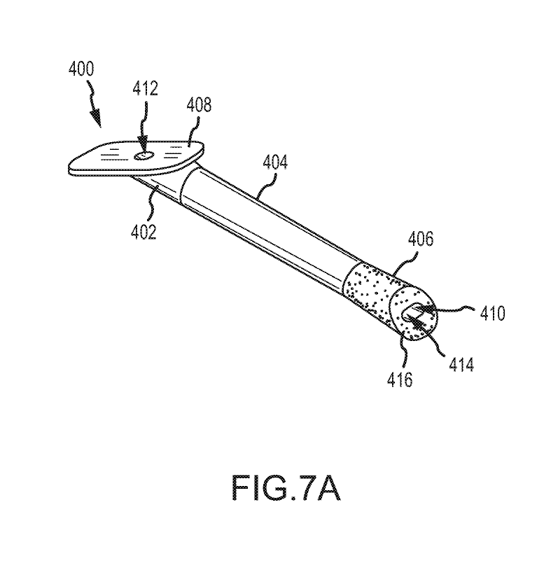

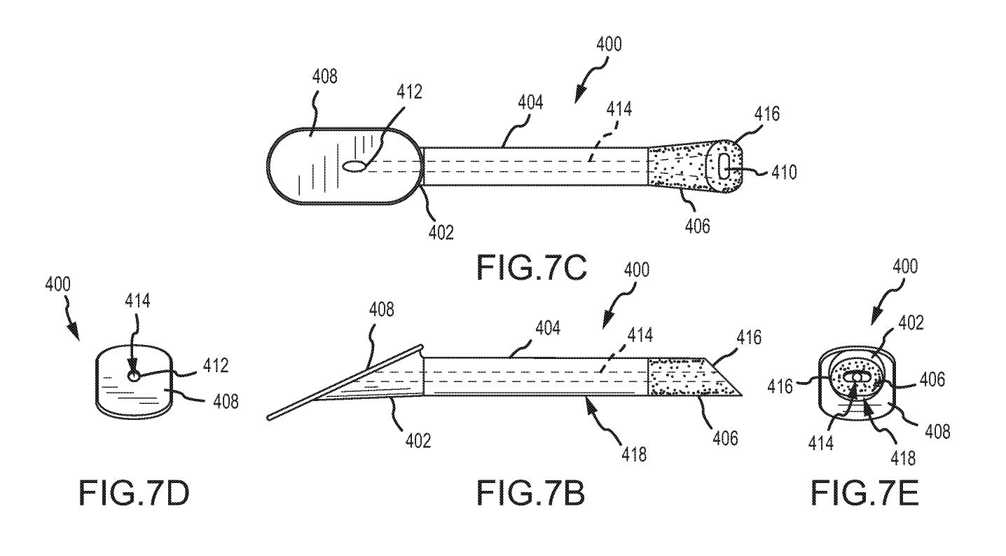

FIGS. 7A-7E depicts another exemplary implementation of a SMP shunt 400 in a deployed/expanded configuration having a proximal portion 402, a distal portion 406, and an elongated middle portion 404 joining the proximal portion 402 and the distal portion 406. An faceplate 408 is formed on the proximal end of the proximal portion 402. In this embodiment, the faceplate 408 is generally rectangular with rounded corners and caps the proximal end 402 at an angle that is acute with respect to its intersection with the longitudinal axis of the shunt 400. As noted above, the faceplate 408 may be formed in any desired shape for best anatomical fit. The angular offset of the faceplate 408 may be determined based upon the anatomical curvature of the eye 2 at the limbus 20 of a particular patient to provide a flat fit of the faceplate 408 against the sclera 16 as further described below. The faceplate 408 should likewise be pliable enough to substantially conform to the sclera 16 so as to avoid erosion through the conjunctiva 18. The faceplate 408 may also be altered by external force to change the shape once the shunt 400 is implanted for improved anatomical fit, to appropriately orient the proximal opening 412 for best drainage, or for more effective delivery of medications. As noted above with respect to other dimensions of the SMP shunts disclosed herein, a number of shunts 400 with faceplates of a variety of angles may be manufactured for “off-the-shelf” use that is most conducive to the anatomy of a particular patient.

The distal portion 406 may be formed with an angular face 416 to create a sharp, lancet-type point. This sharp point may aid in the advancement and penetration of the shunt 400 through the sclera 16 and reduce or obviate the need for a larger incision through the sclera 16 for implantation. The distal portion 406 may further flare lateral to a greater lateral width than the lateral width of the middle portion. This lateral flare may operate as a retention feature to help maintain the distal portion 406 of the shunt 400 within the anterior chamber 22 and counteract possible proximal motion of the shunt 400 within the sclera when implanted.

The distal portion 406 may further be formed with a different formulation of SMP (as indicated by the stippling in the figures) than the proximal portion 402 and the middle portion 404. The SMP formulation of the distal portion 406 may have a higher Tg than the proximal portion 402 and the middle portion 404. In this way the distal portion 406 is not affected by body heat and will remain in a compressed configuration until acted upon by an external energy source to heat it to its higher Tg. In one embodiment, this Tg may be about 10° C. higher than body temperature of 37° C. In an exemplary embodiment, focused light energy of between 750 nm and 900 nm, e.g., as produced by a laser diode, may be used to heat the distal portion. In one particular implementation, a 810 nm laser diode may be used to direct the radiant light energy through the cornea 4 to heat the distal portion 406. While in this exemplary embodiment, the distal portion 406 is formed of the different SMP formulation, it should be understood that any portion (e.g., the faceplate) or portions of a stunt could be formed of alternate SMP formulations to have a different actuation characteristic or other material characteristics, e.g., different rubbery modulus, color, stress or strain, etc. Such alternately formulated portions may be located anywhere along the length of the shunt, coaxially situated, located in laminate fashion, or wound through the other portions.

In an exemplary embodiment, the distal portion 406 may be formulated as 30% tert Butylacrylate (tBA), 60% isobornyl acrylate, 10% PEGDMA 550 and 0.2% 2,2′-Azobis(2-methylpropionitrile) (AIBN) used as a thermal initiator while the proximal portion 402 and middle portion 404 may be formed, for example, of 80% tBA and 20% PEGDMA 550 or any other SMP formulations that create the desired material properties. The distal portion 406 may further be colored to aid in later heating of the distal portion as further described below. In this way, the distal portion 406 may be configured to deploy or expand at slower rate or at a later time (e.g., by the application of additional energy from an external energy source) than the proximal portion 402 and the middle portion 404 as further described below.

The middle portion 404 of the shunt 400 may have a generally curved or oval cross section with a relatively flat bottom wall 418. A lumen 414 extends within the shunt 400 from a proximal opening 412 in the faceplate 408 to a distal opening 410 in the distal portion 406. In this exemplary embodiment, the distal opening 410 is formed as a laterally oblong opening is larger in cross-sectional area than the proximal opening 412. The lumen 414 tapers in cross-sectional area from the distal opening 410 to the proximal opening 412, where it is circular in cross-section normal to the longitudinal axis of the shunt 400, but appears oval on the face of the faceplate 408 due to the angular orientation of the faceplate 408.

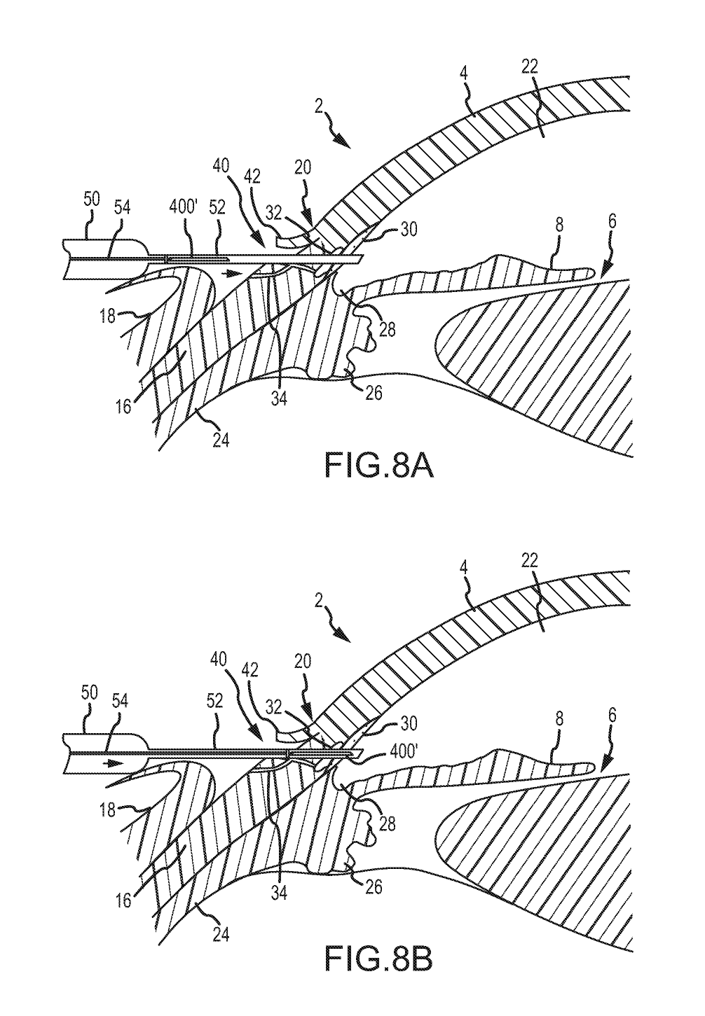

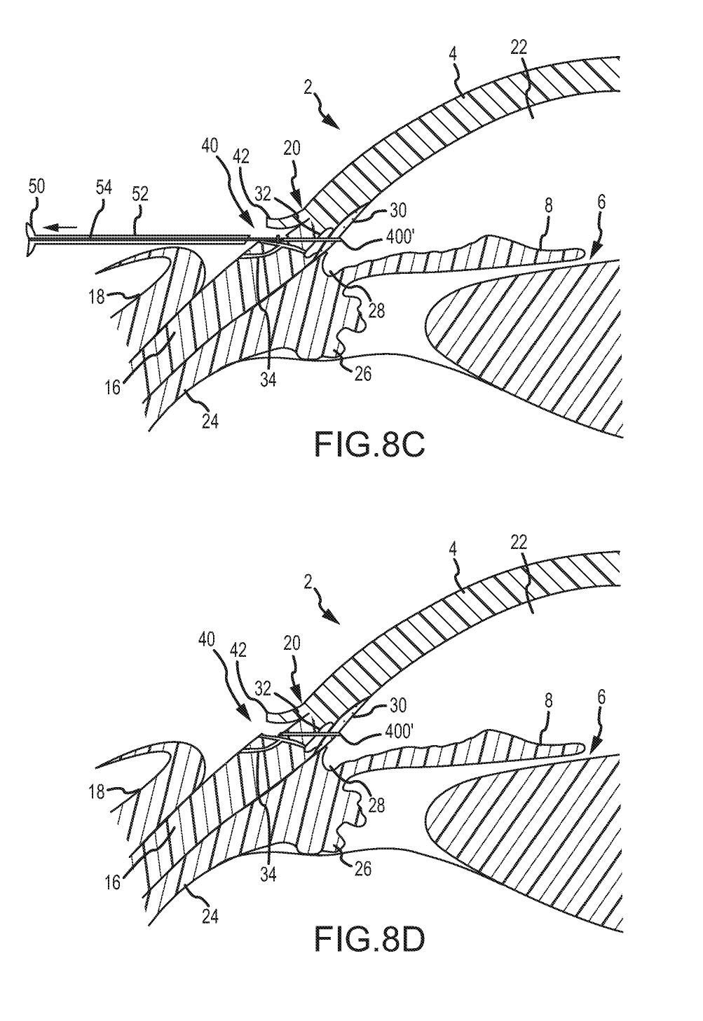

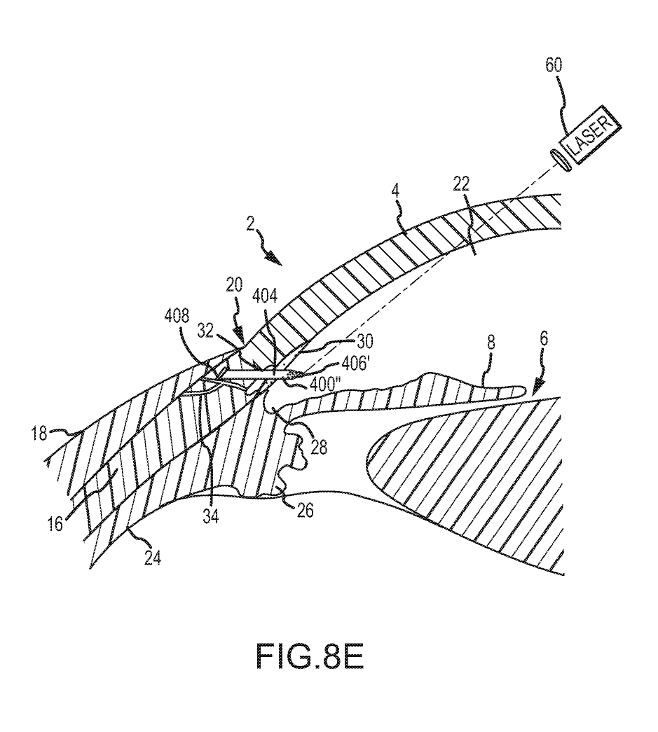

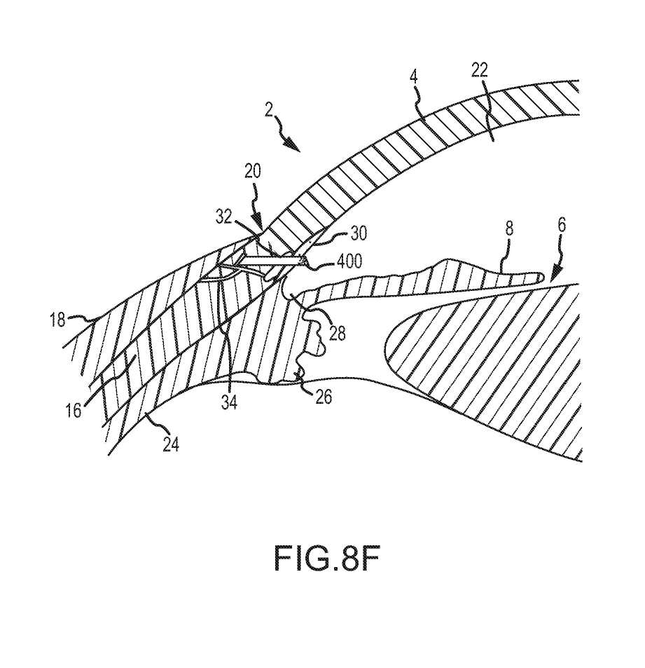

FIGS. 8A-8F depict an exemplary procedure for implantation of the shunt 400 at a location 40 adjacent the limbus 20 through the sclera 16 and into the anterior chamber 22. In other implementations, the shunt embodiments disclosed herein may alternatively be implanted transcorneal for drainage directly across the cornea to the tear film or inferior/superior formix of the eye. As shown in FIG. 8A, the conjunctiva 18 at the limbus 20 may be incised and reflected away from the cornea 4 to reveal the underlying sclera 16. A small patch (e.g., 3-5 mm square) of the surface of sclera 16 adjacent the limbus 20 may also be incised on three edges and approximately 1 mm deep provide a flap 42 to cover faceplate 408 of the proximal portion 402 of the shunt 400 post implantation. An introducer 50 with a needle tip 52 may be advanced under the flap 42 at or slightly posterior to the limbus 20 through the remaining thickness of the sclera 16 in to the anterior chamber 22 at the anterior chamber angle 28. It should be noted that, in some implementations, it may not be necessary or desirable to initially reflect the conjunctiva 18 or both the conjunctiva 18 and the sclera 16 and the needle 52 may be advanced directly through one or both of the conjunctiva 18 and the sclera 16 at the limbus 20 by the introducer 50.

The shunt 400′ in a compressed, predeployed configuration my be placed within the lumen of the needle 52 before the needle is advanced through the sclera 16. In an alternative method, the needle 52 may be positioned in the anterior chamber 22 before the shunt 400′ is placed within the lumen of the needle 52. Once the needle 52 is in position with its tip in the anterior chamber 22, a plunger 54 within the introducer 50 may be used to advance the shunt 400′ within the needle 52 until the shunt 400′ is positioned through the sclera 16 as shown in FIG. 8B. Next the plunger 52 may be held in a fixed position while the introducer 50 is drawn proximally to remove the needle 52 from around the shunt 400′, thereby leaving the shunt 400′ appropriately positioned within the sclera 16 as shown in FIG. 8D. Such a procedure can be considered a one step insertion process. At this point the surgeon may need to manually reposition the shunt 400′ slightly so the that faceplate 408 is proximal to the sclera 16 under the flap 42 and that the distal portion 406 is positioned within the anterior chamber 22.

As the shunt 400′ is exposed to the patient's body heat, the SMP material forming the proximal portion 402 and the middle portion 406 of the shunt 400′ expands to reform the shunt 400″ into a partially deployed shape as shown in FIG. 8E. At this point, the distal portion 406 of the shunt shunt 400″ remains compressed while the faceplate 408 has unfurled and lies flat against the sclera 16 under the flap 42 at the incision location 40. As noted above, in other surgical implementations, the faceplate 408 may alternatively be located under the conjunctiva 18 sitting snugly on the scleral surface if the shunt 400″ is not placed under a scleral flap 42. Also, as shown in FIG. 8E, the middle portion 404 has radially expanded to create a tight friction fit within the incision formed in the sclera 16 by the needle 52 and the lumen 414 therein is fully open. However, the distal portion 406′ formed of the different SMP formulation with a higher Tg remains compressed and the lumen 414 within the distal portion 406′ remains closed as shown in FIG. 8E.

Leaving lumen 414 in the distal portion 406′ closed at the time of initial implantation may be advantageous in the context of post-operative care. It has been found that when aqueous humor drains immediately after implantation of an anterior chamber bypass shunt, healing of the scleral incision may be impeded and scarring of tissue around the faceplate 408 can occur. Further, ocular hypotony immediately, which often offurs after surgery, can be avoided because there is not an immediate outflow of aqueous humor through the stent in addition to seepage through the wound around the stent that drops the intraocular pressure below desirable levels. Once the incision wounds heal (e.g., within 10-14 days) the distal portion can be further heated above its Tg to fully expand the distal portion 406 and open the lumen 414 to allow for drainage of the aqueous humor to the bleb pocket between the conjunctiva 18 and the sclera 16. The increase in temperature can be induced by RF radiation, laser radiation, UV light, or chemical reaction.

In an exemplary embodiment, a near-infrared (e.g., 810 nm) laser diode may be used to activate the SMP functionality of the distal portion 406′ of the shunt 400″ once the scleral incisions have healed. As noted above, the SMP formulation of the distal portion 406 may be colored. This is advantageous for imparting thermal energy to the distal portion 406′ because if the distal portion 406′ were optically clear, the laser energy would merely pass through the SMP material without causing any enhanced heating effect. In one implementation, the SMP formulation of the distal portion 406′ may be colored and have a Tg of approximately 60° C. In alternate embodiments, the entire shunt 400 may be colored. As shown in FIG. 8E, a laser diode may be directed through the cornea 4 to focus heat energy on the distal portion 406′, raise the temperature of the SMP material above 60° C., and thereby activate the SMP material to expand and return to its original “deployed” configuration. In this exemplary implementation, the shunt 400 is thereby fully expanded, the lumen 414 is open along the entire length, and the distal portion 406 forms the wedge structure to further secure the shunt 400 against the interior wall of the sclera 16 at the anterior chamber angle 28.

In an alternate embodiment, rather than forming the distal portion of a different SMP formulation, the lumen in the distal portion may be formed using the same SMP as the proximal portion and the middle portion, but may further be stopped with a plug containing a drug that slowly elutes once implanted to promote healing. For example, a plug in the lumen of the distal portion may elute one or more of the following: an anti-inflammatory agent, an anti-hypertensive agent, an antibiotic agent, a steroid (to decrease scleral scarring), or other agents, or combinations thereof. The drug or other agent may be held within a matrix to form the plug. The plug may slowly disintegrate and elute over time to similarly block the lumen for 10-14 days until the plug has fully disintegrated, leaving the lumen clear for transport of aqueous humor from the anterior chamber 22.

### CONCLUSION

All directional references (e.g., proximal, distal, upper, lower, upward, downward, left, right, lateral, longitudinal, front, back, top, bottom, above, below, vertical, horizontal, radial, axial, clockwise, and counterclockwise) are only used for identification purposes to aid the reader's understanding of the present invention, and do not create limitations, particularly as to the position, orientation, or use of the invention. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. The exemplary drawings are for purposes of illustration only and the dimensions, positions, order and relative sizes reflected in the drawings attached hereto may vary.

The above specification, examples and data provide a complete description of the structure and use of exemplary embodiments of the invention as defined in the claims. Although various embodiments of the claimed invention have been described above with a certain degree of particularity, or with reference to one or more individual embodiments, those skilled in the art could make numerous alterations to the disclosed embodiments without departing from the spirit or scope of the claimed invention. Other embodiments are therefore contemplated. It is intended that all matter contained in the above description and shown in the accompanying drawings shall be interpreted as illustrative only of particular embodiments and not limiting. Changes in detail or structure may be made without departing from the basic elements of the invention as defined in the following claims.

What is claimed is:

1. A shunt for implantation within the sclera between the conjunctiva and the anterior chamber for treatment of glaucoma, the shunt comprising
a tube defining a lumen formed of a shape memory polymer material with a glass transition temperature at or slightly above body temperature that has an initial shape and a deformed shape; wherein
the shunt has a first lumen diameter and a first outer diameter in the deformed shape;
the shunt has a second lumen diameter and a second outer diameter in the initial shape;
the second lumen diameter is greater than the first lumen diameter and the second outer diameter is greater than the first outer diameter; and
the deformed shape is a compressed, predeployed configuration; and
the initial configuration is substantially identical to a post implantation, radially expanded configuration.

2. The shunt of claim 1 further comprising
a first portion formed of the shape memory polymer material; and
a second portion formed of an alternate shape memory polymer material of a different formulation with a Tg high enough above body temperature that body temperature has no effect on the alternate shape memory polymer material.

3. The shunt of claim 2, wherein the second portion has a Tg 10° C. or more higher than body temperature.

4. The shunt of claim 3, wherein the alternate shape memory polymer material comprises a color additive.

5. The shunt of claim 2, wherein the second portion is a distal portion of the shunt that is configured for positioning in the anterior chamber post implantation.

6. The shunt of claim 2, wherein the second portion is deployable from a compressed configuration to an expanded configuration upon application of radiant energy to the second portion between 750 nm and 900 nm.

7. The shunt of claim 1, wherein a distal end of the shunt is formed with a sharp, lancet-like profile.

8. The shunt of claim 1, wherein the lumen further comprises a plurality of substantially parallel lumen.

9. The shunt of claim 1, wherein the second lumen diameter tapers from a wider cross-sectional area at a distal end of the shunt to a narrower cross-sectional area at a proximal end of the shunt.

10. The shunt of claim 1, wherein a proximal end of the shunt is formed as a flat plate that extends as a flange of an area greater than a cross-sectional area of the second outer diameter.

11. The shunt of claim 10, wherein the flat plate is oriented at an acute angle with respect to a longitudinal axis of the shunt.

12. The shunt of claim 1, wherein the SMP material comprises a tert-butyl acrylate (tBA) monomer and a bisphenol A propoxylate diacrylate (BPA-P) diacrylate cross-linking polymer.

13. The shunt of claim 1, wherein the SMP material comprises a tert-butyl acrylate (tBA) monomer and a poly(ethylene glycol) dimethacrylate (PEGDMA) cross-linking polymer.

14. The shunt of claim 1, wherein a one-way valve is provided within the lumen.

15. The shunt of claim 1, wherein a medicament is provided within the lumen, on a surface of the shunt, or within a matrix of the shape memory polymer material.

16. The shunt of claim 2, wherein the second portion is formed to occlude the lumen in the compressed, predeployed configuration.

17. A method for treating glaucoma comprising
implanting a shape memory polymer shunt in a predeployed, compressed configuration substantially at or posterior to the limbus, through the sclera, and into the anterior chamber substantially at the anterior chamber angle whereby a proximal end of the shunt is positioned ab externo and a distal end of the shunt is position ab interno within the anterior chamber; and
activating the shape memory polymer shunt with an external stimulus to transform the shunt into a deployed, expanded configuration that is substantially identical to an original molded configuration.

18. The method of claim 17, wherein
the shunt has a first lumen diameter and a first outer diameter in the predeployed, compressed configuration;
the shunt has a second lumen diameter and a second outer diameter in the deployed, expanded configuration; and
the second lumen diameter is greater than the first lumen diameter and the second outer diameter is greater than the first outer diameter.

19. The method of claim 17, wherein the shunt comprises
a first portion formed of the shape memory polymer material; and
a second portion formed of an alternate shape memory polymer material of a different formulation with a Tg high enough above body temperature that body temperature has no effect on the alternate shape memory polymer; and
wherein the method further comprises
activating the second portion with an alternate external stimulus to transform the second portion into a deployed, expanded configuration that is substantially identical to an original molded configuration.

20. The method of claim 17, wherein the SMP material comprises a tert-butyl acrylate (tBA) monomer and a bisphenol A propoxylate diacrylate (BPA-P) diacrylate cross-linking polymer.

21. The method of claim 17, wherein the SMP material comprises a tert-butyl acrylate (tBA) monomer and a poly(ethylene glycol) dimethacrylate (PEGDMA) cross-linking polymer.

22. The method of claim 17, wherein the shunt, once implanted and transformed, exhibits greater than 98 percent shape recovery from the compressed configuration to the expanded configuration with respect to the original molded configuration.

23. The method of claim 17, wherein activating operation further comprises applying heat above Tg to the shunt.

24. The method of claim 23, wherein Tg is substantially equal to 37° C.

25. The method of claim 17, wherein the activating operation further comprises applying ultraviolet radiation to the shunt.

26. The method of claim 19, wherein the second activating operation further comprises applying radiant energy of between 750 nm and 900 nm to the second portion.

27. The method of claim 19, wherein the alternate shape memory polymer material comprises a color additive.

28. A method of manufacturing an aqueous humor bypass shunt comprising
providing a shape memory polymer material with a Tg slightly greater than or equal to human body temperature;
molding the shape memory polymer material into a permanent tubular shunt form;
mechanically compressing the molded tubular shunt radially at a temperature above Tg to deform the molded tubular shunt into a radially smaller form factor; and
cooling the compressed tubular shunt while still in compression to a temperature below Tg to thereby create a stable compressed tubular shunt with a smaller form factor.

29. The method of claim 28, wherein
the compressed tubular shunt has a first lumen diameter and a first outer diameter in the deformed shape;
the molded tubular shunt has a second lumen diameter and a second outer diameter in the permanent tubular shunt form; and
the second lumen diameter is greater than the first lumen diameter and the second outer diameter is greater than the first outer diameter.

30. The method of claim 28 further comprising
providing an alternate shape memory polymer material of a different formulation with a Tg high enough above body temperature that body temperature has no effect on the alternate shape memory polymer material; and
wherein the molding operation further comprises
simultaneously and contiguously molding a first portion of the permanent tubular shunt form with the shape memory polymer material and molding a second portion of the permanent tubular shunt form with the alternate shape memory polymer material.
